(19) United States
(12) Patent Application Publication
(10) Pub. No.: US 2014/0178323 A1
(43) Pub. Date: Jun. 26, 2014

(54) SALINE STYLING MEDIUM

(71) Applicant: Henkel AG & Co. KGaA, Duesseldorf (DE)

(72) Inventors: Diane Metten, Hamburg (DE); Burkhard Mueller, Duesseldorf (DE); Marie Meisel, Hemslingen (DE)

(73) Assignee: Henkel AG & Co. KGaA, Duesseldorf (DE)

(21) Appl. No.: 14/132,039

(22) Filed: Dec. 18, 2013

(30) Foreign Application Priority Data
Dec. 20, 2012 (DE) 10 2012 223 999.5

(51) Int. Cl. A61K 8/20; A61Q 5/06; A61K 8/81

(52) U.S. Cl. CPC A61K 8/20; A61K 8/8182; A61K 8/8176; A61Q 5/06
USPC 424/70.15

(57) ABSTRACT

Products that, when used, provide naturally curly hairstyles include, based on their weight, 0.1 to 20 wt % setting polymer(s), and 0.01 to 1 mol/kg sodium chloride, and 0.0025 to 1 mol/kg polyvalent metal cation(s) of formula Me^{x+} where x=2 or 3.

### FIELD OF THE INVENTION

The present invention generally relates to hair care agents, and more particularly relates to adjuvants for the temporary deformation of keratinic fibers, in particular human hair.

“Keratinic fibers” are understood in principle as all animal hair, e.g. wool, horsehair, angora wool, furs, feathers, and products or textiles produced therefrom. By preference, however, the keratinic fibers are human hairs.

### BACKGROUND OF THE INVENTION

An attractive-looking hairstyle is generally regarded these days as an indispensable element of a well-groomed appearance. Given the currents of fashion, more and more hairstyles regarded as chic are ones that, for many types of hair, can be constructed, or maintained for a longer period of time of up to several days, only with the use of setting active substances. Hair treatment agents that serve for permanent or temporary shaping of the hair therefore play an important role. While with permanent reshaping, the chemical structure of the keratin-containing fibers is modified by reduction and oxidation, no such modification of the chemical structure takes place in the case of temporary reshaping. Corresponding agents for the temporary deformation usually contain synthetic polymers and/or waxes as a setting active substance. Agents for assisting the temporary reshaping of keratin-containing fibers can be packaged, for example, as a hair spray, hair wax, hair gel, hair foam.

The most important property of an agent for the temporary deformation of keratinic fibers, hereinafter also called a “styling agent,” is to impart the strongest possible hold to the treated fibers in the re-configured shape, i.e. in a shape imposed upon the fibers. If the keratinic fibers involved are human hairs, terms also used are a strong “hairstyle hold” or a high “degree of hold” of the styling agent. The hairstyle hold is determined substantially by the nature and quantity of the setting active substances used, although the further constituents of the styling agent, and the application form, can also have an influence.

A high degree of hold can cause hairstyles to feel “thick”; in extreme cases, consumers complain of “helmet hair.” This is disadvantageous in particular with curly hairstyles, since curling generates a full and gently flowing hairstyle that loses much of its natural beauty as a result of too much thickness and hold.

In biological terms, curling is produced by a change in the distribution of keratinaceous substances in the hair. It is not yet known, however, which variation in the trichohyalin gene produces these changes in the hair cells, and how exactly this procedure occurs. It is certain that the difference with respect to straight hair is produced by a biochemical property of the sheath of the hair root.

Uncurled hair can also be temporarily or permanently curled using suitable agents, so that even people with naturally straight hair can take advantage of curly hairstyles.

It is known that sea salt curls even straight hair, so that even people with straight hair have a “beach look” with curly hair after swimming in seawater. This fact is utilized in innumerable commercially obtainable “seawater sprays” for curling straight hair or for freshening curls.

Since this effect is attributed to the magnesium salt content of seawater, innumerable styling agents also exist that contain magnesium sulfate alongside usual ingredients such as care-providing substances and setting polymers.

Accordingly, it is desirable to make available a styling agent that enables both freshening of curls and curling of straight hair without making the hair too heavy, so that a naturally curly appearance is achieved.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic hair treatment agent includes, based on its weight, 0.1 to 20 wt % setting polymer(s), 0.01 to 1 mol/kg sodium chloride, and 0.0025 to 1 mol/kg polyvalent metal cation(s) of formula Me^{x+} where x=2 or 3.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that the combination of one or more setting polymers with sodium chloride and polyvalent cations results in products that furnish naturally curly hairstyles when they are used.

The subject matter of the present invention is, in a first embodiment, a cosmetic hair treatment agent containing, based on its weight,

a) 0.1 to 20 wt % setting polymer(s), and
b) 0.01 to 1 mol/kg sodium chloride, and
c) 0.0025 to 1 mol/kg polyvalent metal cation(s) of formula Me^{x+} where x=2 or 3.

The agents according to the present invention contain as component a) 0.1 to 20 wt % setting polymer(s). For purposes of the invention, the setting polymer contributes, in the context of the temporary deformation of keratin-containing fibers, to maintaining the shape imposed upon the fibers (in the case of hair, in particular the hold of a hairstyle or the hair volume). The so-called curl retention test is often used as a test method for the setting effect of an active substance.

Preferred agents according to the present invention contain the setting polymers in a total quantity from 0.2 wt % to 17.5 wt %, by preference from 0.5 wt % to 15 wt %, particularly preferably from 2.0 wt % to 10.0 wt %, and in particular from 3.0 to 8.0 wt %, based in each case on the weight of the agent.

Preferred setting polymers are selected from the nonionic setting polymers, anionic setting polymers, amphoteric setting polymers, and cationic setting polymers, as well as mixtures thereof.

Cationic setting polymers comprise at least one structural unit that contains at least one permanently cationized nitrogen atom. “Permanently” cationized nitrogen atoms are to be understood as those nitrogen atoms which carry a positive charge and thereby form a quaternary ammonium compound. Depending on the tertiary amine used, the following groups are known in particular: alkylammonium compounds, alkenylammonium compounds, imidazolinium compounds, and pyridinium compounds.

Agents preferred for purposes of this embodiment contain cationic setting polymers in a quantity from 0.1 wt % to 20.0 wt %, particularly preferably from 0.2 wt % to 10.0 wt %, very particularly preferably from 0.5 wt % to 5.0 wt %, based in each case on the weight of the agent.

The cationic setting polymers can be selected, according to the present invention, from cationic quaternized cellulose derivatives.

Those cationic quaternized celluloses which carry more than one permanent cationic charge in a side chain have proven in general to be advantageous for purposes of the embodiment.

To be emphasized thereamong are, among the cationic cellulose derivatives, those which are manufactured by the reaction of hydroxyethyl cellulose with a dimethyldiallylammonium reactant (in particular dimethyldiallylammonium chloride), optionally in the presence of further reactants. Particularly suitable in turn among these cationic celluloses are those cationic celluloses having the INCI name Polyquaternium-4, which are marketed e.g. under the names Celquat® H 100, Celquat® L 200 by the National Starch company.

Also suitable are those cationic setting polymers which encompass at least one structural unit of formula (I) and at least one structural unit of formula (VI) and optionally at least one structural unit of formula (V)

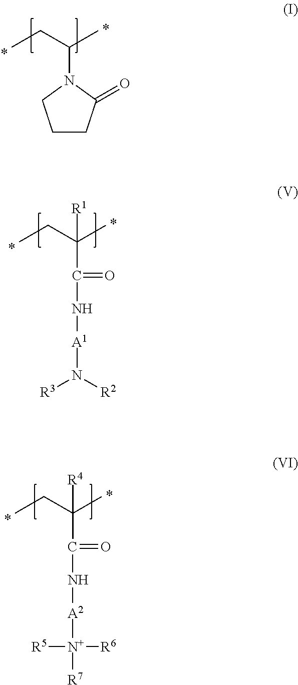

in which
R¹ and R⁴ denote, mutually independently, a hydrogen atom or a methyl group,
A¹ and A² denote, mutually independently, an ethane-1,2-diyl, propane-1,3-diyl, or butane-1,4-diyl group,
R², R³, R⁵, and R⁶ denote, mutually independently, a (C₁ to C₄) alkyl group,
R⁷ denotes a (C₈ to C₃₀) alkyl group.

Suitable compounds are commercially obtainable, for example, as
copolymers of dimethylaminoethyl methacrylate, quaternized with diethyl sulfate, with N-vinylpyrrolidone, having the INCI name Polyquaternium-11, under the designations Gafquat® 440, Gafquat® 734, Gafquat® 755 (each ISP company) and Luviquat PQ 11 PN (BASF SE),
copolymers of methacryloylaminopropyllauryldimethylammonium chloride with N-vinylpyrrolidone and dimethylaminopropyl methacrylamide, having the INCI name Polyquaternium-55, under the commercial names Styleze® W-10, Styleze® W 20 (ISP company),
copolymers of methacryloylaminopropyllauryldimethylammonium chloride with N-vinylpyrrolidone, N-vinylcaprolactam, and dimethylaminopropyl methacrylamide, (INCI name Polyquaternium-69) under the commercial name Aquastyle® 300.

Further serving as setting polymers, particularly preferably usable for purposes of the embodiment, selected from cationic polymers that contain at least one structural unit that comprises a permanently cationized nitrogen atom, are those cationic film-forming and/or cationic setting polymers which comprise at least one structural element of formula (M1)

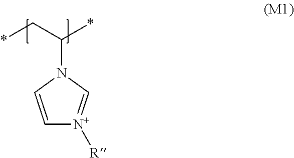

in which
R″ denotes a (C₁ to C₄) alkyl group, in particular a methyl group,
and additionally at least one further cationic and/or nonionic structural element.

It is in turn preferred according to the present invention in the context of this embodiment if at least one copolymer (c1), which encompasses besides at least one structural element of formula (M1) additionally a structural element of formula (I)

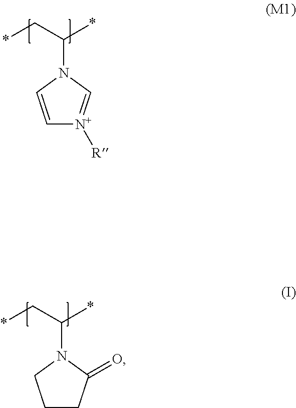

in which
R″ denotes a (C₁ to C₄) alkyl group, in particular a methyl group,
is contained in the polymer used according to the present invention as a cationic setting polymer.

Very particularly preferred cationic setting polymers as copolymers (c1) contain 10 to 30 mol %, by preference 15 to 25 mol %, and in particular 20 mol % structural units according to formula (M1) and 70 to 90 mol %, by preference 75 to 85 mol %, and in particular 80 mol % structural units according to formula (I).

It is particularly preferred in this context if copolymers (c1) contain, besides polymer units that result from incorporation of the aforesaid structural units according to formulas (M1) and (I) into the copolymer, a maximum of 5 wt %, by preference a maximum of 1 wt %, polymer units that are based on the incorporation of other monomers. Copolymers (c1) are by preference constructed exclusively from structural units of formula (M1), where R″=methyl, and (I), and can be described by the general formula (Poly1)

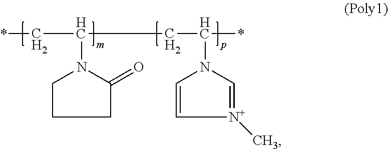

where the indices m and p each vary depending on the molar mass of the polymer and are not intended to signify that these are block copolymers. Structural units of formula (M1) and of formula (I) can instead be present in statistically distributed fashion in the molecule.

If a chloride ion is used to compensate for the positive charge of the polymer of formula (Poly1), these N-methylvinylimidazole/vinylpyrrolidone copolymers are then referred to according to INCI nomenclature as Polyquaternium-16 and are obtainable e.g. from BASF under the commercial names Luviquat® Style, Luviquat® FC 370, Luviquat®FC 550, Luviquat® FC 905, and Luviquat® HM 552.

If a methosulfate is used to compensate for the positive charge of the polymer of formula (Poly1), these N-methylvinylimidazole/vinylpyrrolidone copolymers are then referred to according to INCI nomenclature as Polyquaternium-44 and are obtainable e.g. from BASF under the commercial names Luviquat® UltraCare.

In addition to or instead of the copolymer and/or copolymers (c1), the agents according to the present invention can also contain copolymers (c2) that, proceeding from copolymer (c1), contain as additional structural units those of formula (VII)

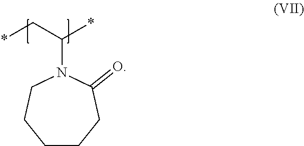

Further particularly preferred agents according to the present invention are thus characterized in that they contain as a cationic film-forming and/or cationic setting polymer at least one copolymer (c2) that contains at least one structural unit according to formula (M1-a) and at least a structural unit according to formula (I) and at least a structural unit according to formula (VII)

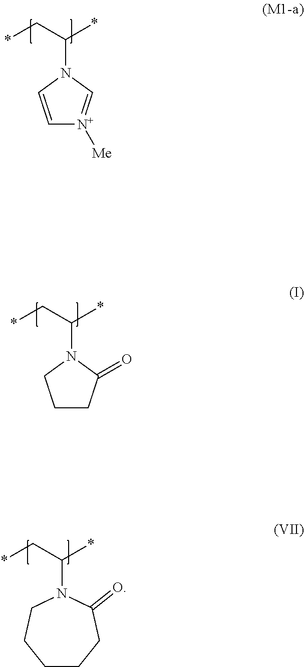

Here as well, it is particularly preferred in the context of this embodiment if copolymers (c2) contain, besides polymer units that result from the incorporation of the aforesaid structural units according to formulas (M1-a), (I), and (VII) into the copolymer, a maximum of 5 wt %, by preference a maximum of 1 wt %, polymer units that are based on the incorporation of other monomers. Copolymers (c2) are by preference constructed exclusively from structural units of formulas (M1-a), (I), and (VII), and can be described by the general formula (Poly2)

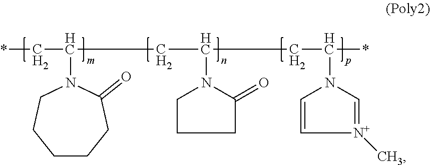

where the indices m, n and p each vary depending on the molar mass of the polymer and are not intended to signify that these are block copolymers. Structural units of the aforesaid formulas can instead be present in statistically distributed fashion in the molecule.

If a methosulfate is used to compensate for the positive charge of the polymer of formula (Poly2), these N-methylvinylimidazole/vinylpyrrolidone/vinylcaprolactam copolymers are then referred to according to INCI nomenclature as Polyquaternium-46 and are obtainable e.g. from BASF under the commercial name Luviquat® Hold.

Very particularly preferred copolymers (c2) contain 1 to 20 mol %, by preference 5 to 15 mol %, and in particular 10 mol % structural units according to formula (M1-a) and 30 to 50 mol %, by preference 35 to 45 mol %, and in particular 40 mol % structural units according to formula (I) and 40 to 60 mol %, by preference 45 to 55 mol %, and in particular 60 mol % structural units according to formula (VII).

In addition to or instead of the copolymer and/or copolymers (c1) and/or (c2), the agents according to the present invention can also contain as a cationic setting polymer copolymers (c3) that comprise, as structural units, structural units of formulas (M-Ia) and (I), as well as further structural units from the group of the vinylimidazole units and further structural units from the group of the acrylamide and/or methacrylamide units.

Further particularly preferred agents according to the present invention are characterized in that they contain, as an additional cationic setting polymer, at least one copolymer (c3) that contains at least one structural unit according to formula (M1-a) and at least a further structural unit according to formula (I) and at least a further structural unit according to formula (VIII) and at least a further structural unit according to formula (IX)

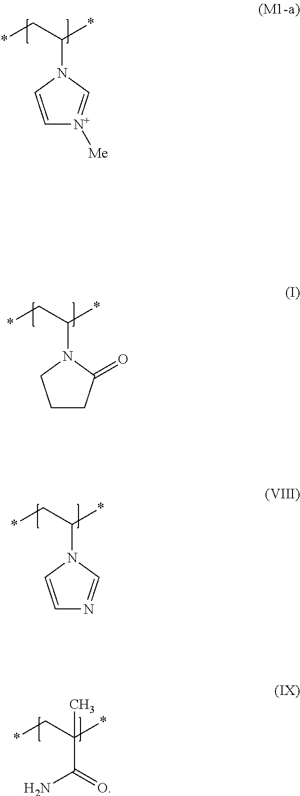

Here as well, it is particularly preferred in the context of this embodiment if copolymers (c3) contain, besides polymer units that result from incorporation of the aforesaid structural units according to formulas (M1-a), (I), (VIII), and (IX) into the copolymer, a maximum of 5 wt %, by preference a maximum of 1 wt %, polymer units that are based on the incorporation of other monomers. Copolymers (c3) are by preference constructed exclusively from structural units of formulas (M1-a), (I), (VIII), and (IX) and can be described by the general formula (Poly3)

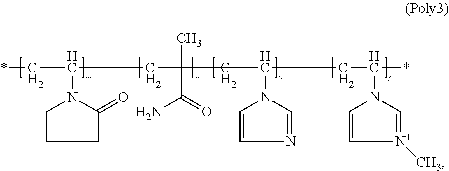

where the indices m, n, o and p each vary depending on the molar mass of the polymer and are not intended to signify that these are block copolymers. Structural units of formulas (M-1a), (I), (VIII), and (IX) can instead be present in statistically distributed fashion in the molecule.

If a methosulfate is used to compensate for the positive charge of the polymer of formula (Poly3), these N-methylvinylimidazole/vinylpyrrolidone/vinylimidazole/methacrylamide copolymers are referred to according to INCI nomenclature as Polyquaternium-68 and are obtainable e.g. from BASF under the commercial name Luviquat® Supreme.

Very particularly preferred copolymers (c3) contain 1 to 12 mol %, by preference 3 to 9 mol %, and in particular 6 mol % structural units according to formula (M1-a) and 45 to 65 mol %, by preference 50 to 60 mol %, and in particular 55 mol % structural units according to formula (I) and 1 to 20 mol %, by preference 5 to 15 mol %, and in particular 10 mol % structural units according to formula (VIII) and 20 to 40 mol %, by preference 25 to 35 mol %, and in particular 29 mol % structural units according to formula (IX).

Among the setting cationic polymers having at least one structural element of the above formula (M1), those considered preferred are:
N-vinylpyrrolidone/1-vinyl-3-methyl-1H-imidazolium chloride copolymers (such as, for example, the one having the INCI name Polyquaternium-16, under the commercial designations Luviquat® Style, Luviquat® FC 370, Luviquat® FC 550, Luviquat® FC 905, and Luviquat® HM 552 (BASF SE)),
N-vinylpyrrolidone/1-vinyl-3-methyl-1H-imidazolium methyl sulfate copolymers (such as, for example, the one having the INCI name Polyquaternium-44, under the commercial designations Luviquat® Care (BASF SE)),
N-vinylpyrrolidone/N-vinylcaprolactam/1-vinyl-3-methyl-1H-imidazolium terpolymers (such as, for example, the one having the INCI name Polyquaternium-46, under the commercial designations Luviquat® Care or Luviquat® Hold (BASF SE)),
N-vinylpyrrolidone/methacrylamide/N-vinylimidazole/1-vinyl-3-methyl-1H-imidazolium methyl sulfate copolymers (such as, for example, the one having the INCI name Polyquaternium-68, under the commercial designation Luviquat® Supreme (BASF SE)),
   as well as mixtures of said polymers.

The agents according to the present invention can contain at least one nonionic setting polymer as a setting polymer. A “nonionic polymer” is understood according to the present invention as a polymer that, in a protic solvent under standard conditions, carries substantially no structural units having permanently cationic or anionic groups that must be compensated for by counter ions to maintain electroneutrality. “Cationic groups” encompass, for example, quaternized ammonium groups but not protonated amines. “Anionic groups” encompass, for example, carboxyl groups and sulfonic-acid groups.

The nonionic setting polymers are contained in the agent according to the present invention of this embodiment preferably in a quantity from 0.1 wt % to 20.0 wt %, particularly preferably from 0.2 wt % to 15.0 wt %, very particularly preferably from 0.5 wt % to 10.0 wt %, based in each case on the weight of the agent according to the present invention.

Those nonionic setting polymers having at least one structural element of formula (M2)

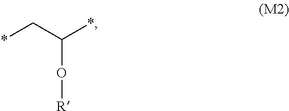

that carry as R′ according to formula (M2) a hydrogen atom, an acetyl group, or a propanoyl group, in particular an acetyl group, are preferably suitable according to the present invention.

The nonionic setting polymers are in turn preferably selected from at least one polymer of the group that is constituted from
homopolymers and nonionic copolymers of N-vinylpyrrolidone,
nonionic copolymers of isobutene.

Suitable polyvinylpyrrolidones are, for example, commercial products such as Luviskol® K 90 or Luviskol® K 85 of the BASF SE company. Suitable polyvinyl alcohols are marketed, for example, under the commercial designations Elvanol® by Du Pont, or Vinol® 523/540 by the Air Products company.

Suitable polyvinyl acetate is marketed, for example, as an emulsion under the trade name Vinac® by the Air Products company.

Agents that contain, as a nonionic setting polymer, at least one polymer selected from the group that is constituted from
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄) alkylacrylamide,
copolymers of N-vinylpyrrolidone with N,N-di(C₁ to C₄) alkylamino-(C₂ to C₄) alkylacrylamide,
   are very particularly preferred according to the present invention.

Particularly preferred agents according to the present invention are characterized in that they contain, based on their weight, 0.15 wt % to 15 wt %, by preference 0.2 wt % to 10 wt %, particularly preferably 0.3 wt % to 5.0 wt %, and in particular 0.35 to 2.5 wt % polyvinylpyrrolidone.

Further particularly preferred agents according to the present invention are characterized in that they contain, based on their weight, 0.2 to 17.5 wt %, by preference 0.25 wt % to 15 wt %, particularly preferably 0.5 wt % to 10.0 wt %, and in particular 1.0 to 8.0 wt % of a copolymer of vinylpyrrolidone and vinyl acetate.

Further possible agents according to the present invention of the embodiment having an additional nonionic setting polymer are characterized in that they contain, as a nonionic setting polymer, at least one copolymer that contains at least a further structural unit according to formula (I) and at least one structural unit according to formula (VII) and at least one structural unit according to formula (VIII)

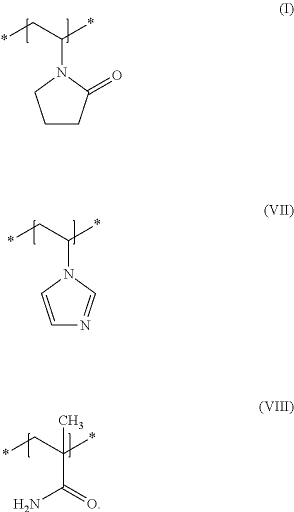

Here as well, it is particularly preferred if these copolymers contain, besides polymer units that result from incorporation of the aforesaid structural units according to formulas (M1-a), (I), (VII), and (VIII) into the copolymer, a maximum of 5 wt %, by preference a maximum of 1 wt %, polymer units that are based on the incorporation of other monomers. Copolymers (c4) are by preference constructed exclusively from structural units of formulas (M1-a), (I), (VII), and (VIII) and can be described by the general formula (Poly4)

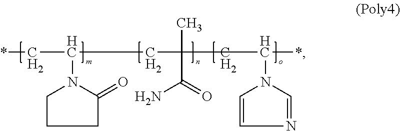

where the indices m, n, o and p each vary depending on the molar mass of the polymer and are not intended to signify that these are block copolymers. Structural units of formulas (I), (VII), and (VIII) can instead be present in statistically distributed fashion in the molecule.

A particularly preferred polymer is selected in this context from the polymers having the INCI name VP/Methacrylamide/Vinyl Imidazole Copolymer, which are obtainable e.g. under the trade name Luviset Clear from the BASF SE company.

The agents according to the present invention can also contain at least one amphoteric setting polymer as a setting polymer. The term “amphoteric polymers” encompasses both those polymers which contain in the molecule both free amino groups and free —COOH or —SO₃H groups and are capable of forming internal salts, and zwitterionic polymers, which contain quaternary ammonium groups and —COO⁻ or —SO₃⁻ groups in the molecule, and those polymers that contain —COOH or —SO₃H groups and quaternary ammonium groups.

An example of an amphoteric setting polymer usable according to the present invention is the acrylic resin obtainable under the name Amphomer®, which represents a copolymer of tert-butylaminoethyl methacrylate, N-(1,1,3,3-tetramethylbutyl)acrylamide, and two or more monomers from the group of acrylic acid, methacrylic acid, and simple alkyl esters thereof.

The latter comprise at least one negatively charged group in the molecule in addition to the cationogenic group and/or positively charged group, and are also referred to as “zwitterionic” polymers.

The amphoteric setting polymers are contained in the agents according to the present invention preferably in quantities from 0.1 to 20 wt %, particularly preferably from 0.05 to 10 wt %, based on the total agent. Quantities from 0.1 to 5 wt % are very particularly preferred.

At least one anionic film-forming polymer can furthermore be used as setting polymers.

Anionic polymers are anionic polymers that comprise carboxylate and/or sulfonate groups. Examples of anionic monomers of which such polymers can be made up are acrylic acid, methacrylic acid, crotonic acid, maleic acid anhydride, and 2-acrylamido-2-methylpropanesulfonic acid. The acid groups can be present in this context entirely or partially as a sodium, potassium, ammonium, mono- or triethanolammonium salt.

Within this embodiment, it can be preferred to use copolymers of at least one anionic monomer and at least one nonionogenic monomer. Reference is made to the substances listed above regarding the anionic monomers. Preferred nonionogenic monomers are acrylamide, methacrylamide, acrylic acid esters, methacrylic acid esters, vinylpyrrolidone, vinyl ethers, and vinyl esters.

Preferred anionic setting polymers are acrylic acid/acrylamide copolymers and in particular polyacrylamide copolymers with sulfonic acid group-containing monomers. A particularly preferred anionic setting copolymer is made up of 70 to 55 mol % acrylamide and 30 to 45 mol % 2-acrylamido-2-methylpropanesulfonic acid, the sulfonic acid group being present entirely or in part as a sodium, potassium, ammonium, mono-, or triethanolammonium salt. This copolymer can also be present in crosslinked form, polyolefinically unsaturated compounds such as tetraallyoxyethane, allylsucrose, allylpentaerythritol, and methylene bisacrylamide preferably being used as crosslinking agents. One such polymer is contained in the commercial product Sepigel® 305 of the SEPPIC company. The utilization of this compound, which besides the polymer component contains a hydrocarbon mixture (C₁₃ to C₁₄ isoparaffin) and a nonionogenic emulsifier agent (Laureth-7), has proven particularly advantageous in the context of the teaching according to the present invention.

The sodium acryloyl dimethyl taurate copolymers marketed, under the designation Simulgel® 600, as a compound with isohexadecane and polysorbate-80 have also proven particularly effective according to the present invention.

Similarly preferred anionic setting homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Further anionic setting polymers usable in preferred fashion are selected from at least one polymer of the group that is constituted from
copolymers of vinyl acetate and crotonic acid (such as those marketed, for example, as a commercial product Aristofiex® A 60, having the INCI name VA/Crotonates Copolymer, by the CIBA company in a 60-wt % dispersion in isopropanol/water),
copolymers of ethyl acrylate and methacrylic acid (such as those marketed, for example, under the trade name Luviflex® Soft with an acid number from 84 to 105, under the INCI name Acrylates Copolymer in an approx. 20- to 30-wt % dispersion in water, by the BASF SE company),
polyurethanes having at least one carboxyl group (such as, for example, a copolymer of isophthalic acid, adipic acid, 1,6-hexanediol, neopentyl glycol, and isophorone diisocyanate, such as the one marketed under the trade name Luviset PUR, having the INCI name Polyurethane-1, by the BASF SE company).

Waxes are preferably used as a setting active substance according to the present invention. Waxes used in the context of the invention are, at 20° C., kneadable, solid to brittle-hard, coarsely to finely crystalline, transparent to opaque but not glass-like; and melt above 40° C. without decomposition. Waxes differ from similar synthetic or natural products (e.g. resins, plastic substances, metal soaps, etc.) in that from 40° C. to 90° C. they transition into the molten, low-viscosity state.

Those waxes which exhibit at 1013 mbar a melting point in the range from 50° C. to 85° C., in particular from 60° C. to 75° C., are preferred according to the present invention.

The waxes are preferably selected from vegetable, animal, and mineral waxes. Waxes particularly preferred according to the present invention are beeswax (cera alba), carnauba wax, candelilla wax, montan wax, microcrystalline waxes (microcrystalline paraffins), and cetyl palmitate. The teaching of the present invention also encompasses the combined use of multiple waxes as component b) of the agent according to the present invention.

The preparations according to the present invention contain waxes by preference in quantities from 1.5 to 60 wt % based on the total preparation. Quantities from 5 to 40 wt %, in particular from 10 to 25 wt %, are particularly preferred.

In summary, agents preferred according to the present invention are characterized in that they contain at least one setting polymer selected from
nonionic polymers based on ethylenically unsaturated monomers, in particular from
homopolymers of N-vinylpyrrolidone,
nonionic copolymers of N-vinylpyrrolidone,
homopolymers and nonionic copolymers of N-vinylcaprolactam,
copolymers of (meth)acrylamide,
polyvinyl alcohol, polyvinyl acetate,
chitosan and derivatives of chitosan,
cationic cellulose derivatives,
cationic copolymers of 3-(C₁ to C₆) alkyl-1-vinylimidazolinium,
homopolymers and copolymers containing the structural unit of formula (M-1)

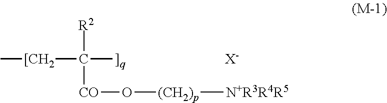

in which R²═—H or is CH₃, R³, R⁴, and R⁵ are selected mutually independently from (C₁ to C₄) alkyl, (C₁ to C₄) alkenyl, or (C₂ to C₄) hydroxyalkyl groups, p=1, 2, 3, or 4, q is a natural number, and X⁻ is a physiologically acceptable organic or inorganic anion,
anionic polymers that comprise carboxylate groups and/or sulfonate groups,
anionic polyurethanes.

As a second essential constituent, the agents according to the present invention contain 0.01 to 1 mol/kg sodium chloride. For a molar mass of NaCl of 58.442769 g/mol, this quantitative indication corresponds to an NaCl content from 0.0584427 to 5.84427 wt % in the agents according to the present invention.

Particularly preferred agents according to the present invention contain, based on their weight, 0.02 to 0.9 mol/kg, by preference 0.03 to 0.75 mol/kg, more preferably 0.04 to 0.6 mol/kg, particularly preferably 0.05 to 0.5 mol/kg, and in particular 0.1 to 0.3 mol/kg sodium chloride.

As a third essential constituent the agents according to the present invention contain 0.0025 to 1 mol/kg polyvalent metal cation(s) of formula Me^{x+} where x=2 or 3, in other words, the corresponding quantity of di- and/or trivalent cations in the form of one or more salts thereof.

Trivalent cations to be considered are in particular Al³⁺ and Fe³⁺; overall, divalent cations are distinctly preferred over trivalent ones, so that preferred agents according to the present invention contain 0.0025 to 1 mol/kg divalent metal cation(s) of formula Me²⁺.

Preferred divalent cations are Mg²⁺, Ca²⁺, Sr²⁺, Mn²⁺, Fe²⁺, Co²⁺, Ni²⁺, Cu²⁺, Zn²⁺, among which Mg²⁺, Mn²⁺, and Zn²⁺ are particularly preferred. Particularly preferred agents according to the present invention accordingly contain 0.0025 to 1 mol/kg divalent metal cation(s) from the group Mg²⁺, Mn²⁺, and Zn²⁺.

Very particularly preferred agents according to the present invention are characterized in that they contain, based on their weight, 0.003 to 0.9 mol/kg, by preference 0.004 to 0.8 mol/kg, more preferably 0.005 to 0.7 mol/kg, particularly preferably 0.0075 to 0.6 mol/kg, and in particular 0.01 to 0.5 mol/kg Mg²⁺.

The agents according to the present invention contain the polyvalent metal cation(s) of formula Me^{x+} where x=2 or 3 by preference in the form of readily soluble salts, so that the metal cations are present in completely dissociated form. Salts particularly preferred for use have solubilities of more than 100 g·l⁻¹ at 20° C., by preference more than 150 g·l⁻¹ at 20° C., more preferably more than 200 g·l⁻¹ at 20° C., and in particular more than 250 g·l⁻¹ at 20° C.

Salts particularly preferred for use are magnesium sulfate heptahydrate MgSO₄ 7H₂O (readily soluble in water: 300 g·l⁻¹ at 20° C.), magnesium chloride MgCl₂ (readily soluble in water: 542 g·l⁻¹ at 20° C.), and magnesium bromide (readily soluble in water: 1015 g·l⁻¹ at 20° C.).

Very particularly preferred agents according to the present invention are characterized in that they contain, based on their weight, 0.25 to 15 wt %, by preference 0.5 to 10 wt %, more preferably 0.75 to 8 wt %, particularly preferably 1 to 7 wt %, and in particular 1.25 to 5 wt % magnesium sulfate heptahydrate.

Agents according to the present invention that contain sodium chloride and magnesium sulfate heptahydrate are extremely preferred in particular when the weight ratio of magnesium sulfate heptahydrate to sodium chloride is 1:2 to 50:1, by preference 1:1 to 25:1, and in particular 2:1 to 10:1.

The agents according to the present invention contain the ingredients and/or active substances in a cosmetically acceptable carrier.

Preferred cosmetically acceptable carriers are aqueous, alcoholic, or aqueous alcoholic media having by preference at least 10 wt % water, calculated on the basis of the total weight of the agent.

The cosmetic carrier according to the present invention particularly preferably contains water, in particular in a quantity such that the cosmetic agent contains, calculated on the basis of the total weight of the agent, at least 10 wt %, in particular at least 20.0 wt %, more preferably at least 40 wt % water.

The alcohols contained can be, in particular, the lower alcohols having 1 to 4 carbon atoms usually used for cosmetic purposes, for example ethanol and isopropanol. The cosmetic carrier can furthermore preferably contain 0.01 to 50 wt %, preferably 0.05 to 45 wt %, and in particular 0.1 to 40 wt % of at least one alcohol that can be selected from ethanol, ethyl diglycol, 1-propanol, 2-propanol, isopropanol, 1,2-propylene glycol, glycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, sorbitol, benzyl alcohol, phenoxyethanol, or mixtures of said alcohols.

The water-soluble alcohols are preferred.

Ethanol, ethyl diglycol, 1-propanol, 2-propanol, isopropanol, 1,2-propylene-glycol, glycerol, benzyl alcohol, and/or phenoxyethanol, as well as mixtures of said alcohols, are particularly preferred.

It is preferred according to the present invention to use at least one (C₁ to C₄) monohydroxyalkane in the agents according to the present invention, in particular in a quantity from 1 to 50 wt %, in particular from 5 to 30 wt %. This is in turn particularly preferred for the formulation as a pump foam or aerosol foam.

Water-soluble solvents particularly preferred as co-solvents are glycerol and/or ethylene glycol and/or 1,2-propylene glycol, in a quantity from 0 to 30 wt % based on the total agent.

It has been found that the effects on straight hair and on stabilization of curls can be even further improved by a polyethylene glycol content. Agents particularly preferred according to the present invention are characterized in that they contain, based on their weight, 0.25 to 10 wt %, by preference 0.5 to 8 wt %, more preferably 0.75 to 6 wt %, particularly preferably 1 to 4 wt %, and in particular 1.5 to 3 wt % polyethylene glycol(s).

Polyethylene glycols that are in particular liquid at room temperature have proven to be particularly suitable in this context, by preference those having molar masses below 600 g/mol, those having molar masses around 400 g/mol being particularly preferred.

Extremely preferred agents according to the present invention contain, based on their weight, 0.25 to 10 wt %, by preference 0.5 to 8 wt %, more preferably 0.75 to 6 wt %, particularly preferably 1 to 4 wt %, and in particular 1.5 to 3 wt % polyethylene glycol(s) of formula (I)

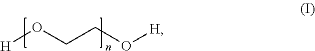

in which n denotes integers from 1 to 14, by preference denotes 4, 5, 6, 7, 8, 9, or 10.

The agents according to the present invention can be formulated in any form usual for cosmetic agents, for example in the form of solutions that can be applied onto the hair as a hair lotion or as a pump or aerosol spray, in the form of creams, emulsions, waxes, gels, or also surfactant-containing foaming solutions or other preparations that are suitable for application to the hair.

Very particularly preferred agents according to the present invention are characterized in that they involve a styling gel, a styling cream, a styling wax, a pump hair spray, an aerosol hair spray, a pump hair foam, an aerosol hair foam, or combinations thereof.

Preferred styling agents are styling gels, pump hair sprays, aerosol hair spray, pump hair foams, and aerosol hair foams.

In the context of the present application, “styling gels” is the general term for clear or opaque products, styling waxes, styling creams, styling lotions, styling jellies, etc. This term ultimately encompasses all agents for the styling of hair that are not hair sprays or foams. “Hair foams” are understood in this context as compositions that form a foam upon removal from a suitable container. It can be necessary to add to the agents ingredients that promote foam formation or stabilize a foam once it has formed. Surfactants and/or emulsifier agents, as already described above, are particularly suitable for this. Surfactants from the group of the cationic surfactants are preferably used.

Hair creams and hair gels as a rule contain structuring agents and/or thickening polymers which serve to impart the desired consistency to the products. Structuring agents and/or thickening polymers are used typically in a quantity from 0.1 to 10 wt %, based on the entire product. Quantities from 0.5 to 5 wt %, in particular 0.5 to 3 wt %, are preferred. Because the polymer combination used according to the present invention has self-thickening properties, however, the addition of further structuring agents and/or thickening polymers is not absolutely necessary. The agents according to the present invention by preference contain no further structuring agents and/or thickening polymers.

If the agents according to the present invention involve an aerosol product, the latter obligatorily contains a propellant. Propellants suitable according to the present invention are, for example, N₂O, dimethyl ether, CO₂, air, and alkanes having 3 to 5 carbon atoms such as propane, n-butane, isobutane, n-pentane, and isopentane, and mixtures thereof. Dimethyl ether, propane, n-butane, isobutane, and mixtures thereof are preferred. The aforesaid alkanes, mixtures of the aforesaid alkanes, or mixtures of the aforesaid alkanes with dimethyl ether are preferably used as the only propellant. The invention also expressly encompasses, however, the concurrent use of propellants of the fluorochlorocarbon type, but in particular the fluorocarbons. For a given spray apparatus, the sizes of the aerosol droplets and/or of the foam bubbles, and the respective size distribution, can be adjusted by way of the quantitative ratio of propellant to the remaining constituents of the preparations.

The quantity of propellant used varies as a function of the specific composition of the agent, the packaging used, and the desired type of product (e.g. hair spray or hair foam). When conventional spray apparatuses are used, aerosol foam products contain the propellant preferably in quantities from 1 to 35 wt % based on the total product. Quantities from 2 to 30 wt %, in particular from 3 to 15 wt %, are particularly preferred. Aerosol sprays generally contain larger quantities of propellant. In this case the propellant is used preferably in a quantity from 30 to 98 wt % based on the total product. Quantities from 40 to 95 wt %, in particular from 50 to 95 wt %, are particularly preferred.

The aerosol products can be manufactured in usual fashion. As a rule all the constituents of the respective agent, with the exception of the propellant, are introduced into a suitable pressure-tight container. The latter is then sealed with a valve. Lastly, the desired quantity of propellant is introduced using conventional techniques.

A second subject of the invention is therefore a method for the temporary deformation of keratinic fibers in which the cosmetic agent according to the present invention is applied onto the hair as a pump hair spray, aerosol hair spray, pump hair foam, aerosol hair foam, styling gel, styling cream, styling wax, or combinations thereof, and is optionally worked into the hair using the palms of the hands and/or the fingers.

The statements made regarding the agents according to the present invention apply mutatis mutandis to the method according to the present invention. The desired deformation of the hair can be brought about using the fingers or hands, and with suitable conventional aids such as a comb or brush.

A third subject of the present invention is the use of the agents according to the present invention for the temporary deformation of keratinic fibers. The agents according to the present invention, and products that contain said agents, are notable in particular for the fact that they impart a very strong hairstyle hold to hair treated with them, without thereby making the hair brittle or inflexible. A pleasant, soft feel is instead achieved.

The agents according to the present invention can be formulated in any form usual for cosmetic agents, for example in the form of solutions that can be applied onto the skin or hair as a face lotion or hair lotion or as a pump or aerosol spray, in the form of creams, emulsions, waxes, gels, or also surfactant-containing foaming solutions or other preparations that are suitable for application to the skin or hair.

### EXAMPLES

The following quantitative indications are to be understood, unless otherwise noted, as percentages by weight. Styling agents E1 to E4 according to the present invention were produced.

| Raw material (INCI name) | E1 | E2 | E3 | E4 |
|---|---|---|---|---|
| Polyvinylpyrrolidone | 0.6 | 2.0 | — | 0.2 |
| PVP/VA Copolymer 60/40 | 3.0 | — | — | — |
| Vinyl Caprolactam/ | — | — | 3.0 | — |
| VP/Dimethylaminoethyl | | | | |
| Methacrylate Copolymer | | | | |
| Polyethylene glycol MG 400 | 2.0 | 3.0 | — | — |
| Propylene glycol | — | — | — | 1.0 |
| Sodium benzoate | — | — | — | 0.2 |
| Sodium chloride | 1.5 | 1.5 | 0.2 | 0.5 |
| Magnesium sulfate heptahydrate | 1.5 | 1.5 | 4.0 | 5.0 |
| PEG-40 Hydrogenated Castor Oil | — | 0.3 | — | 0.5 |
| PEG-60 Hydrogenated Castor Oil | 0.4 | — | — | — |
| D-Panthenol | — | — | 0.5 | — |
| Benzophenone-4 | — | — | 0.2 | — |
| Methylparaben | — | 0.1 | — | — |
| Phenoxyethanol | 0.57 | 0.3 | — | — |
| Dye | + | + | + | + |
| Perfume | + | + | + | + |
| Lactic acid, 80% | 0.01 | 0.03 | — | 0.03 |
| Water, demineralized | to 100 | to 100 | to 100 | to 100 |

The agents were produced by mixing, in usual fashion, the raw materials recited in the table, and were applied onto the hair by spraying the formulations.

The hair had a smooth shine, outstanding hairstyle hold, and excellent curl, but no weighing down of the hairstyle occurred. Using a school grading system, the agents were graded as follows by 10 testers in each case:

| | E1 | E2 | E3 | E4 |
|---|---|---|---|---|
| Average grade | 1.1 | 1.3 | 1.6 | 1.7 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic hair treatment agent comprising, based on its weight,
a) 0.1 to 20 wt % setting polymer(s), and
b) 0.01 to 1 mol/kg sodium chloride, and
c) 0.0025 to 1 mol/kg polyvalent metal cation(s) of formula Me^{x+} where x=2 or 3.

2. The agent according to claim 1, wherein the setting polymers are included in a total quantity from 0.2 wt % to 17.5 wt %, based on the weight of the agent.

3. The agent according to claim 1, wherein the at least one setting polymer is selected from the group consisting of
nonionic polymers based on ethylenically unsaturated monomers, in particular from
homopolymers of N-vinylpyrrolidone,
nonionic copolymers of N-vinylpyrrolidone,
homopolymers and nonionic copolymers of N-vinylcaprolactam,
copolymers of (meth)acrylamide,
polyvinyl alcohol, polyvinyl acetate,
chitosan and derivatives of chitosan,
cationic cellulose derivatives,
cationic copolymers of 3-(C₁ to C₆) alkyl-1-vinylimidazolinium,
homopolymers and copolymers containing the structural unit of formula (M-1) 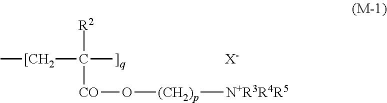
in which R²═—H or is CH₃, R³, R⁴, and R⁵ are selected mutually independently from (C₁ to C₄) alkyl, (C₁ to C₄) alkenyl, or (C₂ to C₄) hydroxyalkyl groups, p=1, 2, 3, or 4, q is a natural number, and X⁻ is a physiologically acceptable organic or inorganic anion,
anionic polymers that comprise carboxylate groups and/or sulfonate groups, and
anionic polyurethanes.

4. The agent according to claim 1, wherein the setting polymer includes, based on the agent's total weight, 0.2 wt % to 17.5 wt % copolymer of vinylpyrrolidone and vinyl acetate.

5. The agent according to claim 1, wherein the setting polymer includes, based on the agent's total weight, 0.15 wt % to 15 wt % polyvinylpyrrolidone.

6. The agent according to claim 1, wherein the sodium chloride is included at a concentration ranging between 0.02 and 0.9 mol/kg.

7. The agent according to claim 1, wherein the polyvalent metal cation(s) include, based on the agent's total weight, 0.003 to 0.9 mol/kg Mg²⁺.

8. The agent according to claim 1, wherein the agent includes, based on its weight, 0.25 to 15 wt magnesium sulfate heptahydrate.

9. The agent according to claim 8, wherein the weight ratio of magnesium sulfate heptahydrate to sodium chloride is 1:2 to 50:1.

10. The agent according to claim 1, wherein the agent includes, based on its weight, 0.25 to 10 wt % polyethylene glycol(s).

11. The agent according to claim 1, wherein the agent includes, based on its weight, 0.25 to 10 wt polyethylene glycol(s) of formula (I) 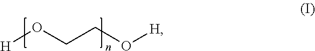
in which n denotes integers from 1 to 14, by preference denotes 4, 5, 6, 7, 8, 9, or 10.
